(19) United States
(12) Patent Application Publication
(10) Pub. No.: US 2008/0017190 A1
(43) Pub. Date: Jan. 24, 2008

(54) Resonating (alerting) metered dose inhaler

(75) Inventors: Aparna Thirumalai Anandampillai, Chennai (IN); Vijayan Thirumalai Anandampillai, Chennai (IN); Anandvishnu Thirumalai Anandampillai, Chennai (IN)

Correspondence Address: APARNA, THIRUMALAI ANANDAMPILLAI, 19, I STREET, PARTHASARATHY NAGAR, ADAM BAKKAM, CHENNAI, 600088, IN

(73) Assignees: APARNA THIRUMALAI ANANDAMPILLAI, CHENNAI (IN); VIJAYAN THIRUMALAI ANANDAMPILLAI, CHENNAI (IN); ANANDVISHNU THIRUMALAI ANANDAMPILLAI, CHENNAI (IN)

(21) Appl. No.: 11/715,298

(22) Filed: Sep. 10, 2007

(30) Foreign Application Priority Data
Dec. 3, 2004 (IN) PCT/IN04/00372
Dec. 3, 2004 (WO) WO 2006 059340

(51) Int. Cl. A61M 11/00; A61M 15/00

(52) U.S. Cl. 128/200.23; 128/203.15

(57) ABSTRACT

This inhaler has a drug vial part and diverging cone shaped mouthpiece (11). Drug vial (7) (with needed drugs in propellant, cosolvents) has a spray rod (8) fitting into a socket (9) with a nozzle (10) leading into the mouthpiece. Mouthpiece (11) has a small air hole (12) with a baffle (13) creating audible sound, for early inhalation triggering. A filter (14) covers the air hole. A dose counter (15) records use. The long mouth piece is kept in mouth, air inhaled, alerting sound is heard, drug vial is pressed, the spray is released at back of throat with longer, deeper and slow inhalation for more lung drug deposit and relief with clean filtered air and dose counter! Modified Insulin (protein) inhaler socket has separate propellant (7) and insulin vials (18)! Insulin vial has a piston (19) and valved exit (20) fitting the socket, before propellant vial. Insulin piston fills needed dose in socket, inhaled, propellant vial is pressed, and insulin spray fills lungs!

### TECHNICAL FIELD

This invention in general relates to medical equipments, specifically a novel kind of metered dose inhaler with a resonating (sound creating) pathway for easy coordination and delivering of drugs to lungs where the drug is absorbed, works and producing relief.

### STATE OF ART

Existing Inhalers used by Asthmatics are metered dose pressure inhalers [called MDI] that send a spray of drug with evaporating propellant and a dry powder inhaler that has no propellant. This device belongs to the pressure inhaler type. Existing MDI inhalers use a multidose vial with drugs suspended in an evaporative medium fitted in a plastic body with a short and wide mouthpiece. The drug mist is released in the short and wide mouthpiece, with a wide divergent mist that coats the mouth and only 5% is sucked into the lungs. The sucking of drug is difficult and wastes the drug, as mouth coating with 5% drug deposit in lungs and 90% failure rate. Dry powder inhalers are also inefficient as drug particles stick together as large poorly absorbed masses with oral deposit that needs water to swallow with 10% deposit in lungs. The metered dose inhalers give 5-micron mist uniformly for easy absorption in lungs and are easy to use than dry powder inhalers. The following description gives critical examination of the inhalers known in the art with its shortcomings. Further in order to over come the problem associated with prior art inhalers, the invention offers the solution to overcome the impediments in the construction and the process of using the inhaler.

Defects in the existing metered dose devices are as follows:
1. Drug spray velocity is fast, allowing too short time for inhalation!
2. Spray of drug starts outside the lip and widens, coating spray into mouth.
3. The larger, wider mouthpiece produces shallow, fast inhalation and allows little time and effective suction for drug deposition in the lungs.
4. Large Mouth Piece is difficult for children and elderly to hold in mouth.
5. Mouth Wastage due to combination of above factors.
6. There is no alert for correct triggering of spray in early inhalation.
7. Drug mist spray diameter is larger [4 cm] than diameter of windpipes [2 cm].
8. Dusty, polluted air is inhaled without a filter that may cause disease to users!
9. There is no simple dose counters.
10. Proteins like insulin are destroyed by drug vial additives and made ineffective.

An extensive search has been carried out using the Internet and related patent specifications were studied for alerting, no mouth coating inhalers. Since the present invention is radically different, the inventor is unable to site any patent specification out of the available databases except WO/03/013634 [also invented by us] on which it is improved. WO/03/013634 is an alerting inhaler with whistle, or visual alert for correct coordination and without air filter. Whistle is loud and good for kids but adults need a softer sound alert without revealing sickness to others. The new invention has a resonating soft sound as air is inhaled for correct coordination without a whistle, has an air filter to clean air and needed further experimentation, changes in structure for optimum spray size and velocity and a novel simple but novel dose counter. Proteins (insulin) can be inhaled with modification.

Ideal inhaler must release a drug spray without coating the mouth. Spray width must be as wide as the windpipe diameter [not more] and of low velocity with longer duration of spray for easy inhalation into lungs. The critical air passages width for slow deep inhalation must be less than 8 mm. A sound at start of inhalation will help in correct trigger and spray release for all drug deposit in lungs. Mouth coating should be avoided with back of mouth release. Existing inhalers are not ideal as the mouthpiece is 3.5 sq.cms in area, no alert, large drug spray diameter of 3-4 cms [wind pipes is 2 cm], difficult to train or use! This invention is easy for children and aged who can now suck deep and vigorously and creates sound for easy coordination, leading to good use of the new inhaler.

Further the invention is addressed to the process of using the new inhaler, which is unique in design and construction, working, use and different.

It is another object of this invention is to invent a novel inhaler which has a lower mouth coating mechanism which helps to deposit the drug only in the lungs correctly.

It is another object of this invention is to invent a novel inhaler with a dose counter and which allows slow and deep inspiration of air, which gives more time for spray creation and spread of spray deeply into the lungs for better drug deposit and relief.

Further objects of the invention will be clear from the ensuring description.

### SUMMARY OF THE INVENTION

The inhaler comprises of a plastic body with a drug vial part and a novel divergent cone shaped long mouthpiece. The drug vial part has a socket for the drug vial and a nozzle for release of spray. The mouthpiece is different from all existing inhalers with a narrow inner end and diverges in a long thin cone such that the drug is deposited in the back of mouth, and propels the drug spray with the inspired air to flow into the lungs.

The air enters the cone shaped mouthpiece in a small hole with a thin baffle across for resonation and has an air filter for trapping dust and germs for safe inhalation. The hole is just small for long, deep, slow inhalation with the air path for easy pressing and release of drug spray. The sound helps in correct start of spray in inhalation. Dose counter is simple.

### STATEMENT OF DRAWINGS

These and other objects and features of the invention will become more apparent upon perusal of following description taken in conjunction with accompanying drawings wherein:

FIG. 1 shows the various components of the novel inhaler according to invention.

FIG. 2 shows the exploded view of the novel inhaler.

FIG. 3 shows the mechanism and spread of the drug mist deep in throat.

FIG. 4 shows a modification with outside air filter for insulin spraying.

FIG. 5 shows in elevation the constructional details of conventional inhaler

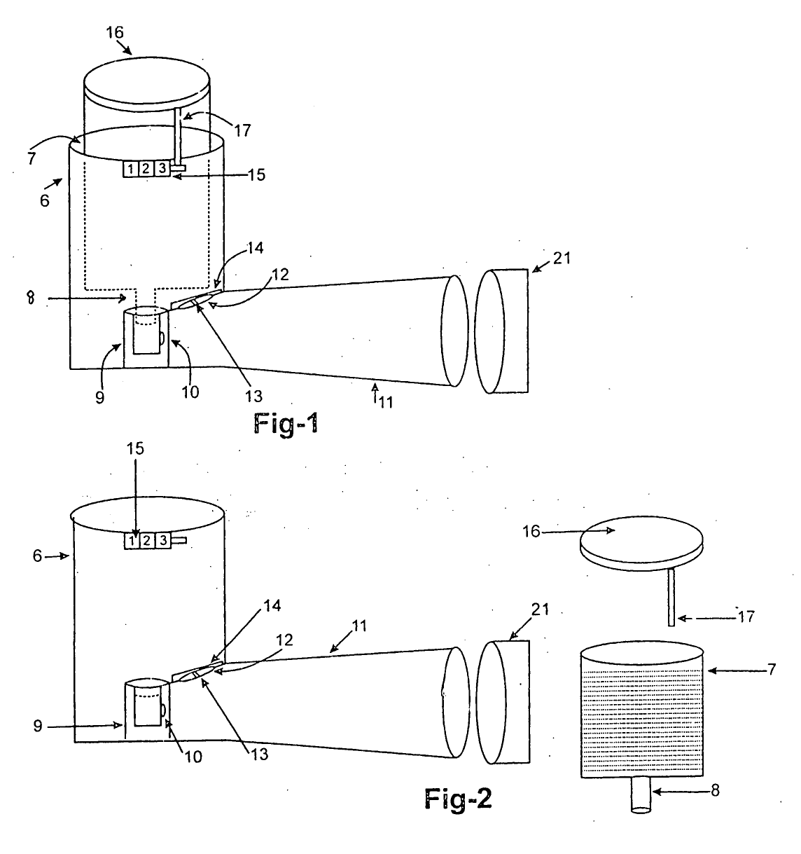

### DESCRIPTION OF PREFERRED EMBODIMENTS

The following specification describes salient features of invention, the method of construction, the method of use and the advantages of the novel invention.

The novel inhaler has a plastic body with a drug vial part and a mouthpiece. The drug vial with needed drug in the vial is fitted to the vial part. The mouthpiece is a longer divergent cone that releases the drug spray at the back of mouth without mouth coating. The mouthpiece has a small hole for airflow to develop deep, long slow inhalation that deposits the drug into the lungs without mouth waste. The mouthpiece directs the spray into wind passages and lungs for good effect. An in built dose counter reveals dose used.

The novel inhaler according to the invention is loaded with drug vial and kept in mouth, air is sucked in through the mouthpiece, a sound is heard which helps to trigger the spray. Drug is now released as a soft spray at the back of mouthpiece, which travels to lungs for better effect.

The novel inhaler according to invention is better because of slow, deeper inspiration breathing through a smaller air hole of mouth piece, long mouth piece that releases drug spray into wind passages without mouth coating unlike existing inhalers, which spray the mouth, need larger suction effort as the drug is sucked from a wider mouth piece (difficult for kids and aged) and do not facilitate deep inspiration for more drug spread in the lungs with no dose counters.

The novel inhaler according to invention needs a lower suction effort by the user to spray (now easy for kids & aged), directs more spray into wind passages without mouth coating and promotes longer and deeper inspiration for the spray to spread to all areas of the lungs through smaller air hole less than 8 mm (for slow deep inspiration) to blow and spray the drug as the air is sucked. The same volume of air sucked through a smaller area takes longer time. The chest muscles also work deeper for forceful inhalation and spread of drug into lungs.

The conventional existing inhaler FIG. 5 consists of a body (1) with a large air inlet (2) at the top in which the drug vial (3) is placed. The body has at the other end has a large area short [less than 2 cms], wide mouthpiece (4). The body on its inside has a spray nozzle (5). To use, the drug can is fitted and the mouthpiece (4) is kept in mouth. Air is inhaled. Inhalation is shallow and fast as the mouthpiece area is large. There is wastage of spray in the mouth as the spray is released outside the lip level and diverges widely. Children and elderly persons do not suck well to create a good the spray release. The device has not been improved for decades.

The new inhaler FIG. 4 comprises of a body (6) preferably of plastic or metal. It has a drug vial (7) with needed drugs and evaporative propelling fluid. The drug vial has a spray rod (8) that fits a socket (9). The socket has a spray nozzle (10) leading to the mouthpiece (11); the nozzle releases the drug spray as a thin slow, low velocity uniform narrow spray. The mouthpiece is shaped as a long divergent cone, and has an air hole 12 with a baffle 13. The hole is less than 8mm for slow, long inhalation. The baffle creates sound as air is sucked for alerting the user to press the drug can and release the spray in early inhalation, so that more drug is sent into lungs for more relief. Baffle 13 creates orderly turbulence to produce a pleasant soft, audible sound that helps to trigger correctly and also help in long inhalation as creation of sound encourages longer deeper inhalation, easy now! A dust cap 21 fits mouthpiece for cleanliness in storage.

The device alerts the user to trigger the device in inspiration, does not spray into mouth, directs flow of mist maximally to air passages delivering correct doses, and is safe for children, aged, even in disorientation! A filter (14) made of paper or plastic mesh and fitting on the hole (12) filters air dust and germs for safe inhalation even in polluted air! A dose counter comprises the counter (15) with three rolling digits fitted to inhaler body. A plate (16) fitted to top of the drug vial with a handle (17) engages the counter on inhaler body and when pressed and moves the counter, showing dose available or used.

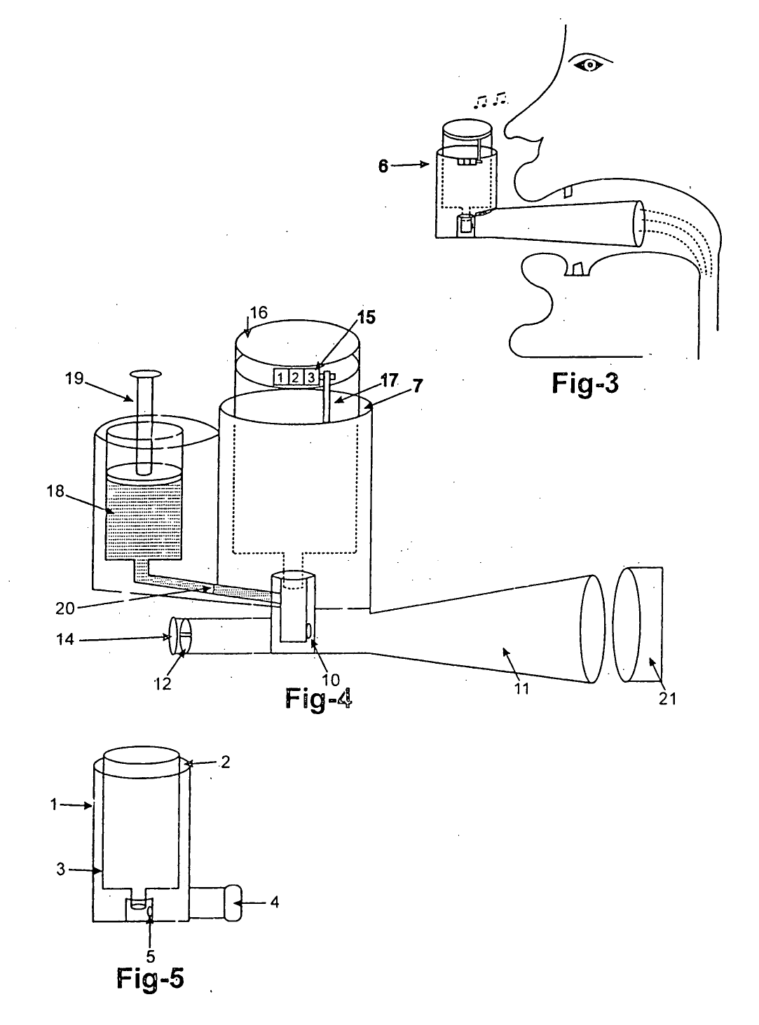

To use, the novel device's mouthpiece is kept between the lips. The air hole of mouthpiece allows air to be sucked in inspiration! The suction of air creates a small sound, which alerts the user to press the drug vial! The sound is a novel indicator of suction of air in inspiration! The drug vial has the drug(s) in solvent with propellant to spray. The mouthpiece is longer and projects into the mouth longer as in FIG. 3. The nozzle produces a mist directed to the windpipe and not to mouth! The drug is carried to distal air passages uniformly, because of deep and slow inhalation. The increased duration of inspiration also helps in spread of mist. The drug is delivered better maximally, without waste, with a visible or audible alarm for the user for the first time in a simple way! When the drug vial is pressed, the shaft 17 strikes the dose counter and moves the counter digits 15, indicating the dose available (200 to 1) or used up (1 to 200).

The inhaler is made of plastic with the orifices, nozzle incorporated as a unit or as separate segments easily assembled.

In FIG. 4, another form of invention for proteins (insulin) shows an air hole (12) is outside, with alerting baffle (13) for a soft whistle. The divergent cone forms the long mouthpiece (11). The propellant vial (7) fits in the body (6), by a spray rod (8), which enters at the socket (9), which has the jet nozzle (10) to back spray in the mouthpiece (11). The socket has an insulin vial (18) with a piston (19) to deposit insulin in the socket. The piston also moves an insulin dose indicator (not shown) when pressed and deposits insulin in the socket. A non-return valve (20) prevents back flow into piston. The air flow is not around the drug vial but by the separate smaller air hole (12)! Thumb steadies the inhaler; other fingers press the drug vial for spray release! A filter (14) removes bacteria and dust of inhaled air, may be used before the air hole to prevent diseases. A dose counter 15 fitted on the drug vial plate (16) is moved by handle (17) engaging the inhaler body rim, if pressed. Counter shows dose used (increasing 1-200) or available (decreasing 200-1). To use, needed dose insulin dose is placed in socket by turning the piston, mouth piece is kept in mouth, inhalation started, sound alerts to press propellant vial, insulin is deposited in lungs! Separation of insulin and propellant is novel and keeps insulin active, as there are no additives to destroy insulin as of now!

The device can be modified. The mouthpiece tube is made as two pieces adjusted on a screw or sliding mechanism. Electronic sensing, spraying and counting are possible but will make the device costly and heavy. Baffle may produce sound of any type. A rotating wheel in a transparent air inlet will also serve as alert to people who do not want sound alert but need visual clue. The body may be a transparent plastic with a handle for easy pressing of the drug vial. Various shapes for body [e.g. oval] and divergent mouthpiece [hexagonal] may be used. A handle for holding and easy pressing can be used. Air hole may be on the mouthpiece outside body. Dose available or used-is indicated.

Drug vial means a unit comprising a can, a crimped cap covering the mouth of the can, and a drug-metering valve situated in the cap, also includes a suitable channeling device, which delivers a predetermined amount of drug formulation upon each activation. The channeling device may comprise, for example, an actuating device for the valve and a cylindrical or cone-like passage through which medicament may be delivered from the filled can via the valve to the mouth of a patient, e.g. a mouthpiece actuator.

The term “drug formulation” means active drug (or a physiologically acceptable solvate thereof) optionally in combination with one or more other pharmacologically active agents such as other anti-inflammatory agents, analgesic agents or other respiratory drugs and optionally containing one or more excipients, and a propellant. The term “excipients” as used herein means chemical agents having little or no pharmacological activity (for the quantities used) but which enhance the drug formulation or the performance of the system. For example, excipients include but are not limited to surfactants, preservatives, flavorings, antioxidants, antiaggregating agents, and cosolvents, e.g., ethanol and diethyl ether.

A polar cosolvent such as C.sub.2-6 aliphatic alcohols and polyols e.g. ethanol, isopropanol and propylene glycol, preferably ethanol, may be included in the drug formulation in the desired amount, either as the only excipient or in addition to other excipients such as surfactants. Suitably, the drug formulation may contain 0.01 to 5% w/w based on the propellant of a polar cosolvent e.g. ethanol, preferably 0.1 to 5% w/w e.g. about 0.1 to 1% w/w.

Drug formulation for use in the invention may, if desired, contain one or more other pharmacologically active agents, selected from any suitable drug useful in inhalation therapy. Medicaments may be selected from, for example, sildenafil for pulmonary hypertension, analgesics, e.g. codeine, dihydromorphine, ergotamine, fentanyl or morphine; anginal preparations, e.g. diltiazem; antiallergics, e.g. cromoglycate, ketotifen or nedocromil; antiinfectives e.g. cephalosporins, pentamidine; antihistamines, e.g. methapyrilene; anti-inflammatories, e.g. beclomethasone, fluticasone propionate, flunisolide, budesonide, tipredane or triamcinolone acetonide; antitussives, e.g. noscapine; bronchodilators, e.g. salbutamol, salmeterol, ephedrine, adrenaline, fenoterol, formoterol, isoprenaline, albuterol, metaproterenol, phenylephrine, phenylpropanolamine, pirbuterol, reproterol, rimiterol, terbutaline, isoetharine, tulobuterol, orciprenaline,or(−)-4-amino-3,5-dichloro-.alpha.[[[6-[2-(2-pyridinyl)ethoxy]hexyl]amino]methyl]benzenemethanol; diuretics, e.g. amiloride; anticholinergics e.g. ipratropium, atropine or oxitropium; hormones, e.g. cortisone, hydrocortisone or prednisolone; xanthines e.g. aminophylline, choline theophyllinate, lysine theophyllinate or theophylline; and therapeutic proteins and peptides, e.g. insulin or glucagon and genetic fragments or anti cancer drugs or any such lung absorbable drugs. It will be clear to a person skilled in the art that, where appropriate, the medicaments may be used in the form of salts (e.g. as alkali metal or amine salts or as acid addition salts) or as esters (e.g. lower alkyl esters) or as solvates (e.g. hydrates) to optimise the activity and/or stability of the medicament and/or to minimize the solubility of the medicament in the propellant.

Drug formulations for Asthma may contain fluticasone propionate (or a physiologically acceptable solvate) in combination with a bronchodilator such as salbutamol (e.g. as the free base or the sulphate salt) or salmeterol (e.g. as the xinafoate salt). Combinations for the other diseases as sildenafil for pulmonary hypertension, insulin for diabetes, luprolide for prostrate cancer etc may be used for treatment.

“Propellants” mean pharmacologically inert liquids with boiling points from about room temperature (25.degree. C.) to about −25.degree. C. which singly or in combination exert a high vapor pressure at room temperature. Upon activation of the drug vial, the high vapor pressure of the propellant in the MDI forces a metered amount of drug formulation out through the metering valve. Then the propellant very rapidly vaporizes dispersing the drug particles. The propellants used in the present invention are low boiling fluorocarbons, HFA, etc.

Drug combinations for use in the invention may be free or substantially free of formulation excipients e.g. surfactants and cosolvents etc. and are advantageous since they may be substantially taste and odour free, less irritant and less toxic than excipient-containing formulations. Thus, a preferred drug formulation consists of fluticasone propionate, or it's physiologically acceptable salt, optionally in combination with one or more other pharmacologically active agents particularly salmeterol (e.g. in the form of the xinafoate salt), and a propellant.

Drug formulations for use in the invention may be free or substantially free of surfactant. Most often the drug vial can and cap are made of aluminum or an alloy of aluminum, although other metals not affected by the drug formulation, such as stainless steel, an alloy of copper or tin plate, glass or plastic may be used.

The drug metering valve consists of parts usually made of stainless steel, a pharmacologically inert and propellant resistant polymer, such as acetal, polyamide (e.g., Nylon.RTM.), polycarbonate, polyester, fluorocarbon polymer (e.g., Teflon.RTM.) or a combination of these materials. Additionally, seals and “O” rings of various materials (e.g., nitrile rubbers, polyurethane, acetyl resin, fluorocarbon polymers), or other elastomeric materials are employed in and around the valve. Particularly preferred coatings for inside of drug vial are pure PFA, FEP and blends of PTFE and polyethersulphone (PES).

The particle size of the particular (e.g., micronised) drug should be less than 20 microns, and, in particular, in the range of 1-10 microns, e.g., 1-5 microns.

It will be apparent to those skilled in the art that modifications to the invention described herein can readily be made without departing from the spirit of the invention. Protection is sought for all the subject matter described herein including any such modifications.

### ADVANTAGES OF THE NEW INVENTION

1. Slow and deep inhalation, as air is drawn through a smaller air hole.
2. The device gives more time to trigger the spray, as the inhalation is slow and long.
3. There is an alerting sound to help time the trigger in early inhalation.
4. There is no mouth coating and waste of drug spray.
5. Useful for kids, old, who can now coordinate the spray mechanism easily.
6. The spray is released at the back of the mouth, which easily goes into windpipes and lungs for good effect.
7. The filter removes all air polluting dust and germ particles for safe inhalation.
8. Dose counter reveals the dose used or available.

We claim

1) A resonating inhaler comprising of a plastic body(6) with a drug vial part and a long, cone shaped mouthpiece(l 1), the said mouthpiece having a small 8mm air hole(12) with a baffle across(13), the said drug vial part having a socket(9) with a spray nozzle(10) opening into the said mouthpiece, the said socket having the drug vial(7) with needed drugs, the said small air hole develops deep and longer inspiration, the said baffle of the air hole makes sound alert for the drug spray trigger, the said spray rod(8) of the drug vial back sprays into the mouth piece and into wind passages, such that the said drug spray with the inspired air flows into the lungs, a dose counter(15) in body records each use.

2) A resonating inhaler as claimed in claim 1, wherein the said air hole has an air dust filter (14), the said filter traps dust particles and germs and cleans the entering air.

3) A resonating inhaler as claimed in claim 1, wherein the said spray path comprises of a drug spray vial (7) with a spray rod (8), the said spray rod fitting a socket (9) in the said body, the said socket having a spray jet nozzle (10) projecting into the said mouthpiece for the spray to be formed and directed back through the mouthpiece into lungs.

4) A resonating inhaler as claimed in claim 1, wherein the said mouthpiece (11) comprises of a long diverging cone with a smaller end at the plastic body and larger end projecting out till middle of mouth, the smaller end having the jet nozzle (10) of the said drug vial socket in its inside, the said mouth piece back spraying the drug spray into the wind passages for correct spray.

5) A resonating inhaler as claimed in claim 1, wherein the said drug vial is placed at right angles to the mouthpiece.

6) A resonating inhaler as claimed in claim 1, wherein the said air hole(12) of the said mouthpiece is less than 8mm and has a baffle across to create an audible sound for correct spray triggering in early inhalation.

7) A resonating inhaler as claimed in claim 1, wherein the inhaler is made of transparent plastic for visual clue.

8) A resonating inhaler as claimed in claim 1, wherein the drug vial comprises of strong metal can, with a crimped cap covering the mouth of the can and a drug-metering valve in the cap.

9) A resonating inhaler as claimed in claim 1, wherein the drug formulation comprises of an active agent for the medical need, as a microns particle, in a solvent, with a propellant with or without surfactants or excipients, the said drug when sprayed reaches the lung for rapid relief.

10) A resonating inhaler as claimed in claim 1, wherein a plate(16) with a dosage counter( 15) is fixed to the drug vial top, the said counter having three digit display, the said counter is rotated by a handled 7) engaging the inhaler body rim, when pressed.

11) A resonating inhaler as claimed in claim 1, wherein the dosage counter(15) is fixed to the body(6), the said counter having three digit display, the said counter is rotated by a handle(17) attached to a plate(16) fixed to drug vial and engaging the counter, when pressed.

12) A resonating inhaler as claimed in claim 1, wherein the inhalation drug may be single or combination of active pharmacological agents as described in the specification.

13) A resonating inhaler for proteins (such as insulin) as claimed in claim 1, wherein the drug and propellant vials are separate, the said insulin vial( 18) has a body with a piston(19) at the top and a lower nozzle with a non return valve(20) fitting a socket, the said socket also having a propellant vial(7), the said insulin piston has an insulin dose indicator when pressed, the said propellant vial has a cover plate(16) with another inhaler dose counter(15), the socket having the spray nozzle(10) in the mouth piece(11), to spray the needed insulin dose, with separate insulin and inhalation dose indicators.
